(19) United States
(12) Patent Application Publication
(10) Pub. No.: US 2016/0151175 A1
(43) Pub. Date: Jun. 2, 2016

(54) CONTROLLED CORONAL STIFFNESS PROSTHETIC ANKLE

(71) Applicant: UNIVERSITY OF WASHINGTON THROUGH ITS CENTER FOR COMMERCIALIZATION, Seattle, WA (US)

(72) Inventors: Glenn KLUTE, Seattle, WA (US); Jeffrey GORGES, Seattle, WA (US)

(21) Appl. No.: 14/905,482

(22) PCT Filed: Aug. 21, 2014
(86) PCT No.: PCT/US14/52003

§ 371 Date: Jan. 15, 2016

Related U.S. Application Data
(60) Provisional application No. 61/868,426, filed on Aug. 21, 2013.

(51) Int. Cl. A61F 2/66; A61F 2/70

(52) U.S. Cl. CPC A61F 2/6607; A61F 2/70; A61F 2002/701

(57) ABSTRACT

A prosthetic ankle device and methods are provided herein to adjust stiffness of the prosthetic ankle. The prosthetic ankle including a base, and a vertical support having a first end pivotally coupled to the base. The prosthetic ankle may also include a first cantilever beam spring having a fixed end coupled to the base such that a length of the first cantilever beam spring in an unloaded position is substantially parallel to a longitudinal axis of the vertical support when (he vertical support is in a neutral position. The prosthetic ankle may also include a stiffness regulator movably coupled to the vertical support. The stiffness regulator may include a platform that defines a first contact surface adjacent the first cantilever beam spring. In addition, the stiffness regulator may pivot with the vertical support relative to the base.

### CROSS-REFERENCE TO RELATED APPLICATIONS

This application claims the benefit of the filing date of U.S. Provisional Patent Application Ser. No. 61/868,426, filed Aug. 21, 2013, which is hereby incorporated by reference in its entirety.

### GOVERNMENT RIGHTS

This invention was made with government support under W81XWH-09-2-0144 awarded by the Department of Defense. The government has certain rights in the invention.

### BACKGROUND

Unless otherwise indicated herein, the materials described in this section are not prior art to the claims in this application and are not admitted to be prior art by inclusion in this section.

Lower limb prosthetic devices may significantly improve quality of life for amputees. However, one area of concern for lower limb amputees with prosthetic devices may be falling. Amputees have a number of factors working against them that make them more likely to fall than the average person. And the fear of falling can have a significant impact on how an amputee uses a prosthetic device and the types of terrain they feel comfortable traversing. One area that may affect user confidence in a prosthetic device is stability. Stability is a function of how the prosthetic device interfaces with the residual limb of an amputee, as well as how the prosthetic device interfaces with a ground surface. For flat, level terrain, the motion of the prosthetic device is more predictable and an amputee typically has more confidence traversing such surfaces. However, on uneven terrain, balance may become more of an issue.

### SUMMARY

Example devices and methods described herein may allow adaptation of a prosthetic ankle to invert and evert to varying degrees depending on the characteristics of the terrain being traversed. For example, the devices and methods described herein may mimic the functions of a normal foot using an adjustable stiffness in the coronal plane. Inversion and eversion may be achieved in the disclosed devices and methods via a stiffness regulator adjustably positioned relative to one or more vertically positioned cantilever beam springs, as described in more detail below, to allow for adjusting stiffness of the prosthetic ankle within each step to react to changing terrain conditions. As such, the disclosed devices and methods may improve walking, running, and other forms of bipedal motion for amputees on uneven terrain. Improving the natural motion of a prosthetic device and the confidence of a user while traversing uneven terrain would improve the quality of life of amputees. Robotic prostheses may also benefit from the mechanical adaptations of the invention to make them more versatile and functional.

Thus, in one aspect, a prosthetic ankle is provided including (a) a base, (b) a vertical support having a first end pivotally coupled to the base, (c) a first cantilever beam spring having a fixed end coupled to the base such that a length of the first cantilever beam spring in an unloaded position is substantially parallel to a longitudinal axis of the vertical support when the vertical support is in a neutral position and (d) a stiffness regulator movably coupled to the vertical support including a platform that defines a first contact surface adjacent the e first cantilever beam spring, where the stiffness regulator may pivot with the vertical support relative to the base.

In a second aspect, a method is provided for adjusting stiffness of a prosthetic ankle. The method may include (a) receiving, by a computing device, sensor data from one or more sensors of a prosthetic ankle, where the prosthetic ankle includes (i) a vertical support having a first end pivotally coupled to a base, (ii) a cantilever beam spring having a fixed end coupled to the base such that a length of the first cantilever beam spring in an unloaded position is substantially parallel to a longitudinal axis of the vertical support when the vertical support is in a neutral position and (iii) a stiffness regulator movably coupled to the vertical support, (b) determining, via the computing device and based on the sensor data, a vertical position of the stiffness regulator relative to the base and one or more of an applied load on the cantilever beam spring and an angular position of the vertical support relative to the base and (c) adjusting a stiffness of the prosthetic ankle.

In a third aspect, another method is provided for adjusting stiffness of a prosthetic ankle. The method may include (a) receiving, by a computing device, sensor data from one or more sensors of a prosthetic ankle, where the prosthetic ankle includes (i) a vertical support having a first end pivotally coupled to abuse, (ii) a cantilever beam spring having a fixed end coupled to the base such that a length of the first cantilever beam spring in an unloaded position is substantially parallel to a longitudinal axis of the vertical support when the vertical support is in a neutral position and (iii) a stiffness regulator movably coupled to the vertical support, (b) tracking, via the computing device and based on the sensor data, a gait cycle of the prosthetic ankle, (c) determining, via the computing device and based on the sensor data, a phase of the gait cycle and (d) adjusting a stiffness of the prosthetic ankle based on the determined phase of the gait cycle.

In a fourth aspect, a non-transitory computer readable memory having stored therein instructions executable by a computing device to cause the computing device to perform functions is described. The functions may include (a) receiving sensor data from one or more sensors of a prosthetic ankle, where the prosthetic ankle includes (i) a vertical support having a first end pivotally coupled to a base, (ii) a cantilever beam spring having a fixed end coupled to the base such that a length of the first cantilever beam spring in an unloaded position is substantially parallel to a longitudinal axis of the vertical support when the vertical support is in a neutral position and (iii) a stiffness regulator movably coupled to the vertical support, (b) determining, based on the sensor data, a vertical position of the stiffness regulator relative to the base and one or more of an applied load on the cantilever beam spring and an angular position of the vertical support relative to the base and (c) adjusting a stiffness of the prosthetic ankle.

In a fifth aspect, a non-transitory computer readable memory having stored therein instructions executable by a computing device to cause the computing device to perform functions is described. The functions may include (a) receiving sensor data from one or more sensors of a prosthetic ankle, where the prosthetic ankle includes (i) a vertical support having a first end pivotally coupled to a base, (ii) a cantilever beam spring having a fixed end coupled to the base such that a length of the first cantilever beam spring in an unloaded position is substantially parallel to a longitudinal axis of the vertical support when the vertical support is in a neutral position and (iii) a stiffness regulator movably coupled to the vertical support, (b) tracking, based on the sensor data, a gait cycle of the prosthetic ankle, (c) determining, based on the sensor data, a phase of the gait cycle and (d) adjusting a stiffness of the prosthetic ankle based on the determined phase of the gait cycle.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A is a perspective view of a prosthetic ankle, according to an example embodiment.

FIG. 1B is a perspective view of example components of the prosthetic ankle of FIG. 1A, according to an example embodiment.

FIG. 1C is a from view of another prosthetic ankle, according to an example embodiment.

FIG. 1D is a perspective view of example components of the prosthetic ankle of FIG. 1A, according to an example embodiment.

FIG. 1E is a perspective view of example components of the prosthetic ankle of FIG. 1A, according to an example embodiment.

FIG. 2 is a block diagram illustrating example functional architecture of components of a prosthetic ankle.

FIG. 3 is a flowchart illustrating an example method for adjusting stiffness of a prosthetic ankle.

FIG. 4 is a flowchart illustrating another example method for adjusting stiffness of a prosthetic ankle.

FIG. 5 depicts a computer-readable medium configured according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

As used herein, with respect to measurements, “about” means +/−5%.

As used herein, “coronal plane” is a longitudinal plane that is perpendicular to a length of a prosthetic foot.

As used herein, “inversion” means a movement that tilts a bottom surface of a prosthetic foot towards a midline of a body.

As used herein, “eversion” means a movement that tilts a bottom surface of a prosthetic foot away from a midline of a body.

As used herein, a “neutral position” with respect to a vertical support means that the vertical support is substantially vertical and is neither inverted nor everted relative to a midline of a body.

As used herein, a “gait cycle” includes a heel strike phase, a mid-stance phase, a toe-off phase and an unloaded swing phase. A heel strike phase begins when a heel of a prosthetic foot contacts the ground and transitions into a mid-stance phase as the prosthetic foot rolls forward along the ground bringing the whole foot in contact with the ground and then transitions into a toe-off phase in which the foot continues to roll forward lifting the heel from the ground such that the ball of the foot and toes remain in contact with the ground and finally lifts the prosthetic foot completely from the ground in a swing phase.

Lower limb prosthetic devices provide amputees with increased mobility and an improved quality of life. Increased stability and balance of such lower limb prosthetic devices is important for user confidence in traversing a variety of terrain. A major point of balance for both above knee and below knee amputees is the ankle and foot, where the limb interacts with the terrain. Example devices described herein allow adaptation of a prosthetic ankle to invert and evert to varying degrees depending on the need related to the terrain being traversed. The devices described herein may mimic the functions of a normal ankle and foot using an adjustable stiffness in the coronal plane. In particular, inversion and eversion may be permitted in the disclosed devices and methods due to one or more vertically positioned cantilever beam springs, including a moveable stiffness regulator on the cantilever beam springs to allow for adjusting stiffness of the prosthetic ankle within each step to react to changing terrain conditions. Such a configuration may improve walking, running, and other forms of bipedal motion for amputees on uneven terrain.

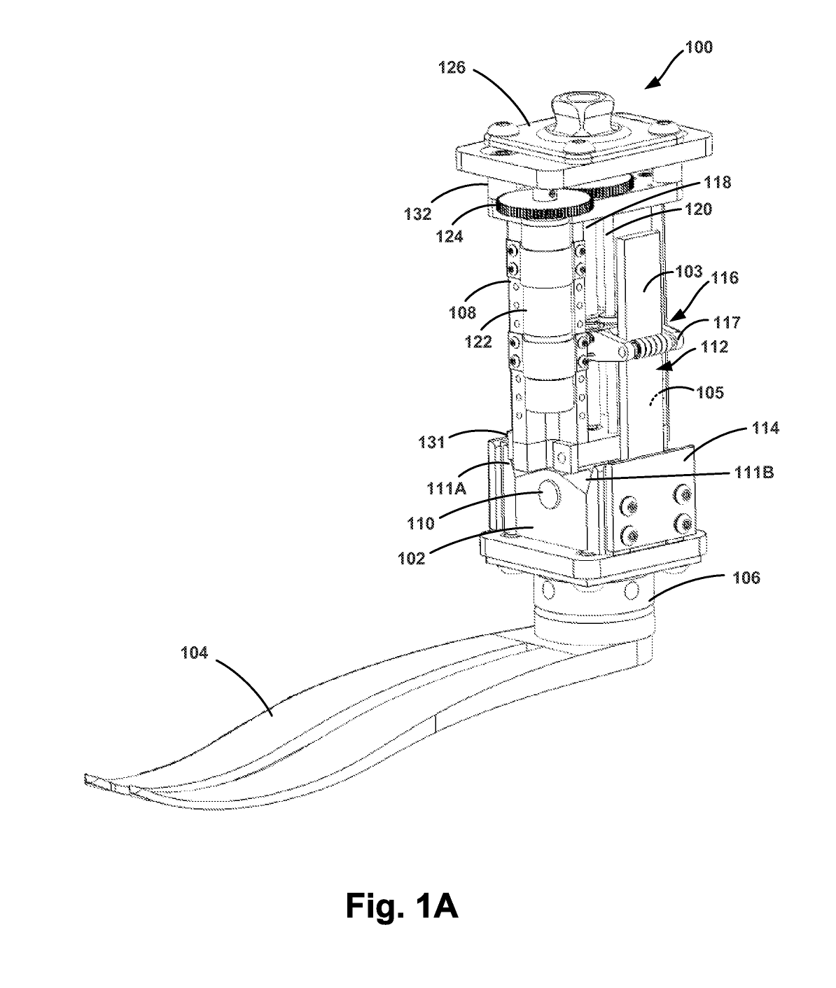

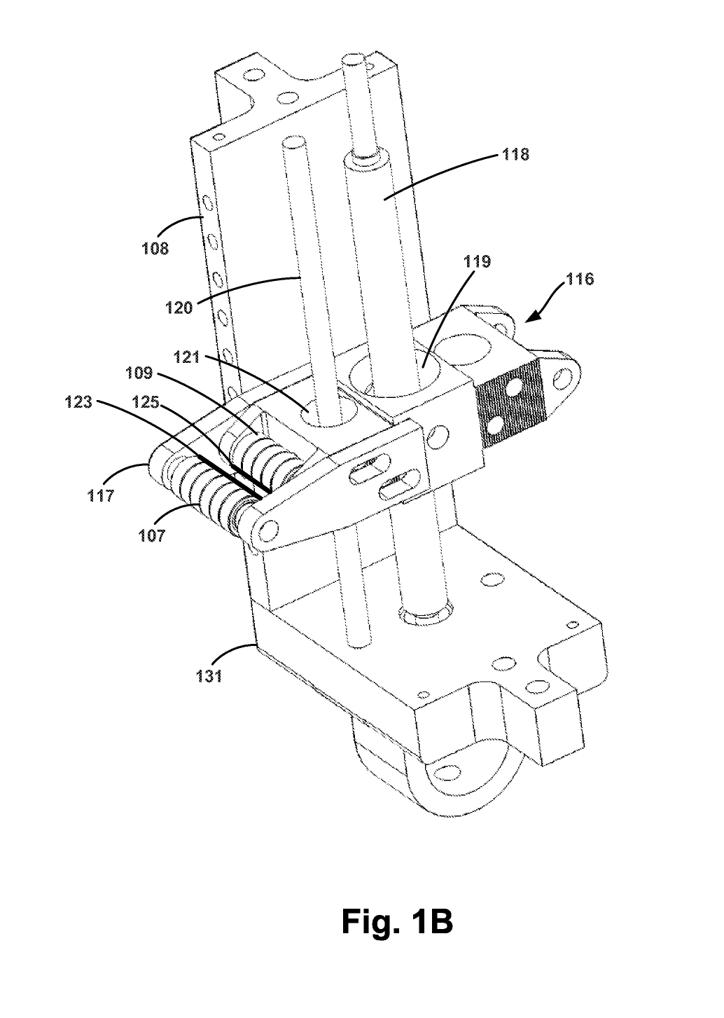

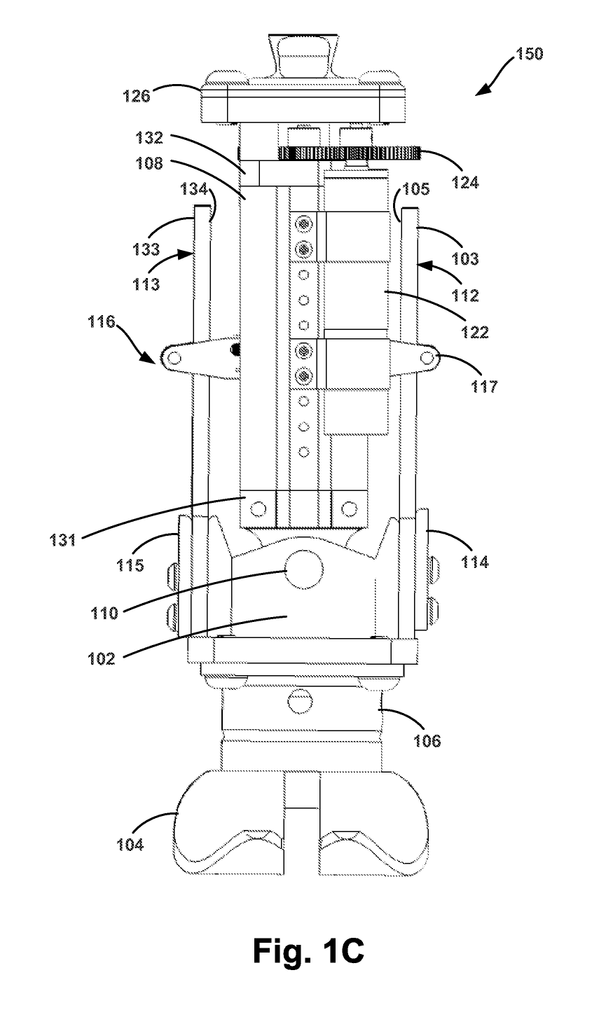

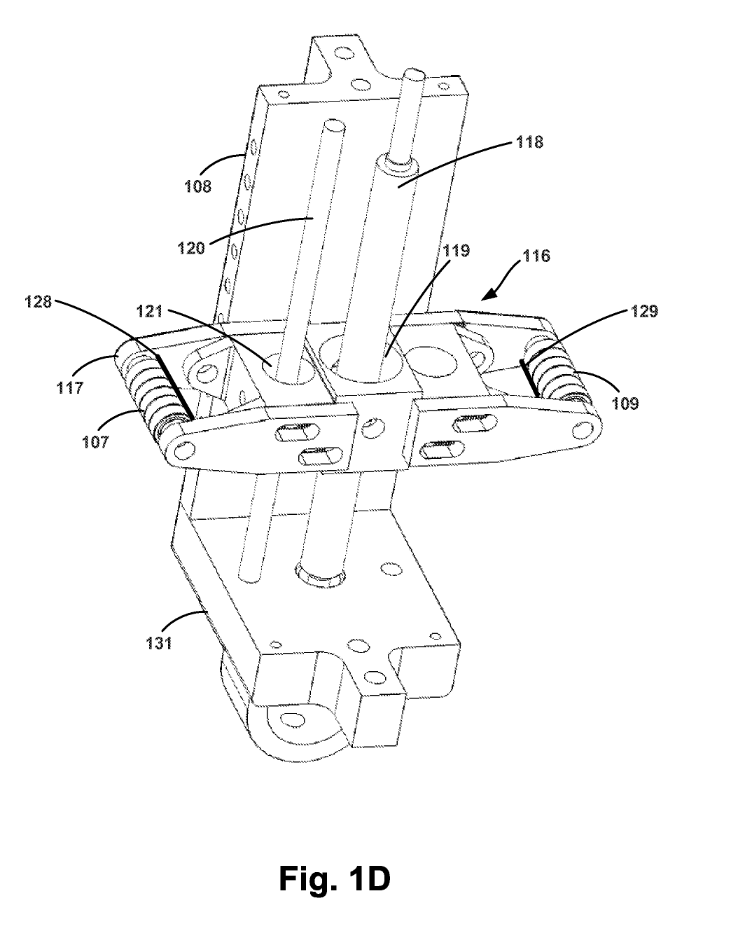

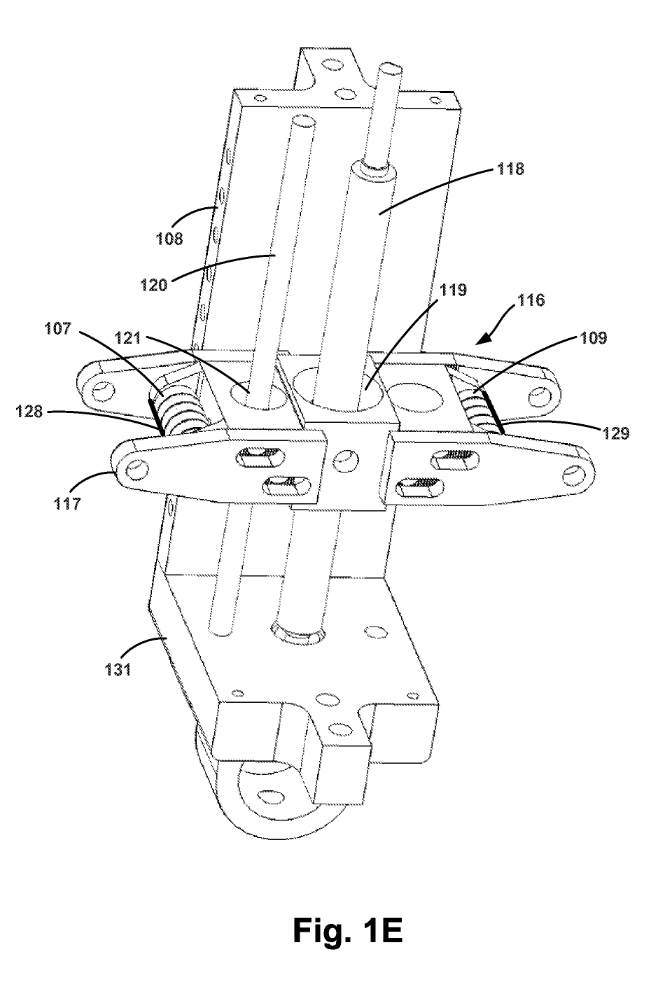

In a first aspect, FIG. 1 illustrates an example prosthetic ankle 100 in accordance with one embodiment of the invention. The prosthetic ankle 100 may include a base 102 that may be configured coupled to a prosthetic foot 104 via a pyramid adapter 106, for example. The prosthetic ankle 100 may also include a vertical support 108 having a first end pivotally coupled to the base 102. As shown in FIG. 1, the vertical support 108 may be coupled to the base 102 via a pin joint 110. In other examples, the vertical support 108 may be coupled to the base 102 via a hinge joint, a four bar linkage, or a ball and socket joint. Other example pivotally coupling mechanisms are contemplated as well.

The prosthetic ankle 100 may further include a cantilever beam spring 112 having a fixed end coupled to the base 102. The cantilever beam spring 112 may be positioned on a medial or lateral side of the prosthetic ankle 100. The length of the cantilever beam spring 112 may range from about 30 mm to about 120 mm. The cantilever beam spring 112 may include carbon fiber material, plastic, metal, or a combination of materials. Further, the cantilever beam spring 112 may include a variable profile, such that a width of the beam varies along its length. The cantilever beam spring 112 may be fixed to the base 102 with a clamp 114 that may be adjustable to secure cantilever beam springs of varying thicknesses to the base 102. The cantilever beam spring 112 may be positioned such that a length of the cantilever beam spring 112 in an unloaded position is substantially parallel to a longitudinal axis of the vertical support 108 when the vertical support 108 is in a neutral position with respect to the base 102.

The prosthetic ankle 100 may also include a stiffness regulator 116 movably coupled to the vertical support 108. As shown in FIGS. 1A-1B, the stiffness regulator 116 may include a platform 117 that defines a first contact surface 123 adjacent an outer surface 103 of the cantilever beam spring 112, and a second contact surface 125 adjacent an inner surface 105 of the cantilever beam spring 112. The first contact surface 123 and the second contact surface 125 may include a plurality of bearings 107, 109 to reduce friction as the stiffness regulator 116 moves with respect to the cantilever beam spring 112.

In operation, the vertical support 108 pivots relative to the base 102 about the pin joint 110, for example, in a coronal plane of the prosthetic ankle 100. The stiffness regulator 116 pivots with the vertical support 108 relative to the base 102. The prosthetic ankle 100 may include a hard stop 111A, 111B to either side of the pin joint 110, such that the vertical support 108 may pivot a maximum of about twenty-five degrees from the longitudinal axis of the vertical support 108 in both an inverted direction and an everted direction. As the vertical support 108 pivots in relation to the base 102, a point load is applied to the cantilever beam spring 112 via the first contact surface 123 and the second contact surface 125, depending on the direction of rotation of the vertical support 108 relative to the base 102.

In particular, the prosthetic ankle 100 may be a left ankle, such that the cantilever beam spring 112 is positioned on a lateral side of the prosthetic ankle 100. In such an embodiment, when the prosthetic ankle 100 is evening, the second contact surface 125 applies a point load to the inner surface 105 of the cantilever beam spring 112. When the prosthetic ankle 100 is inverting, the first contact surface 123 applies a point load to the outer surface 103 of the cantilever beam spring 112. In another embodiment, the prosthetic ankle 100 may be a right ankle, such that the cantilever beam spring 112 is positioned on a medial side of the prosthetic ankle 100. In such an embodiment, when the prosthetic ankle 100 is everting, the first contact surface 123 applies a point load to the outer surface 103 of the cantilever beam spring 112. When the prosthetic ankle 100 is inverting, the second contact surface 125 applies a point load to the inner surface 105 of the cantilever beam spring 112.

As discussed above, the stiffness regulator 116 is movably coupled to the vertical support 108. In one example, the prosthetic ankle 100 may include a threaded rod 118 coupled to the first end 131 of the vertical support 108, and further coupled to a second end 132 of the vertical support 108. The stiffness regulator 116 may include a threaded nut 119 that is centrally disposed in the platform. The threaded nut 119 may be coupled to the threaded rod 118 such that the threaded rod 118 extends through the platform 117 of the stiffness regulator 116. In such a configuration, when the threaded rod 118 rotates, the accompanying threaded nut 119 fixed to the stiffness regulator 116 is driven either up or down depending on the direction of the rotation. The prosthetic ankle 100 may further include a guide rod 120 coupled to the first end 131 and the second end 132 of the vertical support 108. The guide rod 120 may be disposed through a through-hole 121 in the platform 117 of the stiffness regulator 116. The guide rod 120 may further include one or more linear bearings coupled to the platform of the stiffness regulator 116, so as to minimally affect the linear motion of the stiffness regulator 116. The guide rod 120 may be configured to keep the stiffness regulator 116 aligned with the vertical support 108, and prevent the stiffness regulator 116 from rotating as the threaded rod 118 rotates. In another embodiment, the vertical support 108 may include a track to receive rollers coupled to the stiffness regulator 116, such that the rollers transfer the rotational motion of the threaded rod 118 to a linear motion of the stiffness regulator 116 relative to the vertical support 108. Other configurations to prevent the rotation of the stiffness regulator 116 are possible as well.

As the stiffness regulator 116 moves between the first and second ends of the vertical support, the distance from the fixed end of the cantilever beam spring 112 to the point at which the load is applied by the first contact surface 123 or second contact surface 125 changes. The same load applied at different positions will cause different amounts of deflection in the cantilever beam spring 112. Different deflections of the cantilever beam spring 112 will in turn relate to different angular deflections of the prosthetic ankle 100. In particular, moving the stiffness regulator 116 in a direction away from the base 102 decreases stiffness of the prosthetic ankle 100, while moving the stiffness regulator 116 in a direction toward the base 102 increases the stiffness of the prosthetic ankle.

A rotation of the threaded rod 118 may be induced in a number of ways. In one example, a hand crank may be used to rotate the threaded rod 118 to manually adjust the height of the of the stiffness regulator 116. In such an example, the vertical support 108 may include a visual indicator of the position of the platform of the stiffness regulator 116 used to indicate a stiffness setting to a user.

In another example, a motor 122 may be coupled to the threaded rod 118 to induce a rotational motion of the threaded rod 118. In one example, the motor 122 may be coupled to a gear 124 used to transfer the rotational motion of the motor 122 to a complimentary gear fixed to the threaded rod 118. As such, the motor 122 may be configured to rotate the threaded rod 118 relative to the stiffness regulator 116 to move the stiffness regulator 116 along the threaded rod 118 between the first end 131 and the second end 132 of the vertical support 118.

FIG. 1A further illustrates a four-hole pyramid adapter 126 coupled to the second end 132 of the vertical support 118. A prosthetic limb, such as a shank, may be coupled to the four-hole pyramid adapter 126. A bottom portion of the prosthetic limb may include a connector portion that is configured to mate with the four-hole pyramid adapter 126, as an example. Other connection mechanisms are possible as well.

FIG. 1C illustrates a front view of another example prosthetic ankle 150. In particular, prosthetic ankle 150 may include many of the features of prosthetic ankle 100 described above in relation to FIGS. 1A-1B, but may further include a second cantilever beam spring 113. The second cantilever beam spring 113 may be fixed to the base 102 via a second clamp 115 that may be adjustable to secure cantilever beam springs of varying thicknesses to the base 102. The second cantilever beam spring 113 may be positioned such that a length of the second cantilever beam spring 113 in an unloaded position is substantially parallel to the longitudinal axis of the vertical support 108 when the vertical support 108 is in a neutral position with respect to the base 102. Further, the vertical support 108 may be positioned between the first cantilever beam spring 112 and the second cantilever beam spring 113 such that the first and second cantilever beam springs 112, 113 arc arranged on medial and lateral sides of the vertical support 108.

The prosthetic ankle 150 may also include a stiffness regulator 116 movably coupled to the vertical support 108. As shown in FIGS. 1C and 1D, the stiffness regulator 116 may include a platform 117 that defines a first contact surface 128 adjacent an outer surface 103 of the first cantilever beam spring 112, and a second contact surface 129 adjacent an outer surface 133 of the second cantilever beam spring 113. The first contact 128 surface and the second contact surface 129 may include a plurality of bearings 107, 109 to reduce friction as the stiffness regulator 116 moves with respect to the cantilever beam springs 112, 113.

In operation, the vertical support 108 pivots relative to the base 102 about the pin joint 110, for example, in a coronal plane of the prosthetic ankle 100. The stiffness regulator 116 pivots with the vertical support 108 relative to the base 102. As the vertical support 108 pivots in relation to the base 102, a point load is applied to either the first cantilever beam spring 112 via the first contact surface 128 or the second cantilever beam spring 113 via the second contact surface 129.

In particular, the prosthetic ankle 150 may be a left ankle, such that the first cantilever beam spring 112 is positioned on a lateral side of the prosthetic ankle 150, and the second cantilever beam spring 113 is positioned on a medial side of the prosthetic ankle 150. In such an embodiment, when the prosthetic ankle 150 is everting, the second contact surface 129 applies a point load to the outer surface 133 of the second cantilever beam spring 113. When the prosthetic ankle 150 is inverting, the first contact surface 128 applies a point load to the outer surface 103 of the first cantilever beam spring 112. In another embodiment, the prosthetic ankle 150 may be a right ankle, such that the first cantilever beam spring 112 is positioned on a medial side of the prosthetic ankle 150, and the second cantilever beam spring 113 is positioned on a lateral side of the prosthetic ankle 150. In such an embodiment, when the prosthetic ankle 150 is everting, the first contact surface 128 applies a point load to the outer surface 103 of the first cantilever beam spring 112. When the prosthetic ankle 150 is inverting, the second contact surface 129 applies a point load to the outer surface 133 of the second cantilever spring 113.

In another example, as shown in FIG. 1E, the first contact surface 128 may be adjacent an inner surface 105 of the first cantilever beam spring 112, and the second contact surface 129 may be adjacent an inner surface 134 of the second cantilever beam spring 113. As described above, the prosthetic ankle 150 may be a left ankle, such that the first cantilever beam spring 112 is positioned on a lateral side of the prosthetic ankle 150, and the second cantilever beam spring 113 is positioned on a medial side of the prosthetic ankle 150. In such an example, when the prosthetic ankle 150 is everting, the first contact surface 128 applies a point load to the inner surface 105 of the first cantilever beam spring 112. When the prosthetic ankle 150 is inverting, the second contact surface 129 applies a point load to the inner surface 134 of the second cantilever beam spring 113. As described above, in another example the prosthetic ankle 150 may be a right ankle, such that the first cantilever beam spring 112 is positioned on a medial side of the prosthetic ankle 150, and the second cantilever beam spring 113 is positioned on a lateral side of the prosthetic ankle 150. In such an embodiment, when the prosthetic ankle 150 is everting, the second contact surface 129 applies a point load to the inner surface 134 of the second cantilever beam spring 113. When the prosthetic ankle 150 is inverting, the first contact surface 128 applies a point load to the inner surface 105 of the first cantilever beam spring 112.

In another example embodiment, the prosthetic ankle 150 may include a second stiffness regulator (not shown) coupled to the second cantilever beam spring 113. In such an embodiment, the first stiffness regulator 116 may be controlled independent of the second stiffness regulator, such that the stiffness on the medial side of the prosthetic ankle 150 may be different than the stiffness on the lateral side of the prosthetic ankle 150. Other embodiments are possible as well.

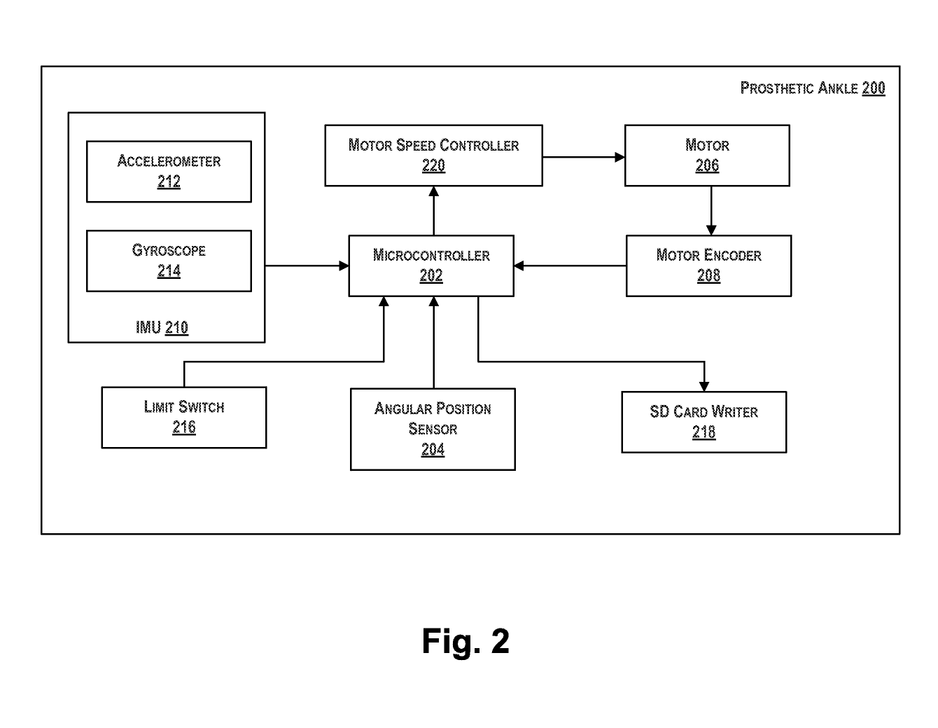

FIG. 2 is a block diagram illustrating example functional architecture of components of a prosthetic ankle 200. The prosthetic ankle 200 may include the features of prosthetic ankle 100 and/or prosthetic device 150 described above in relation to FIGS. 1A-1B. As shown in FIG. 2, the prosthetic ankle 200 may include a microcontroller 202 configured to receive one or more inputs from various components of the prosthetic ankle 200 and to provide outputs to one or more devices. The microcontroller 202 may include at least one processor (which may in turn include at least one microprocessor) that executes instructions stored in a non-transitory computer readable medium. The microcontroller 202 may also represent a plurality of computing devices that may serve to control individual components or subsystems of the prosthetic ankle 200 in a distributed fashion.

In particular, the prosthetic ankle 200 may include an angular position sensor 204 that may detect an angular position of the prosthetic ankle by measuring the angle of the base in relation to the vertical support. The position of the stiffness regulator may be determined by determining the number of rotations of the motor 206 using a motor encoder 208. The motor encoder 208 may relate the rotations of the motor 206 to rotations of the threaded rod based on known gear ratios between the two components. Using the determined angular position of the prosthetic ankle 200 and the position of the stiffness regulator, applied moments and stiffness of the prosthetic ankle 200 may be determined by the microcontroller 202 based on known properties of the cantilever beam spring.

Another input to the microcontroller 202 is an inertial measurement unit (IMU) 210. The IMU 210 may include both an accelerometer 212 and a gyroscope 214, which may be used together o determine the orientation, position, and/or velocity of the prosthetic ankle 200. The accelerometer 212 can measure the orientation of the prosthetic ankle 200 with respect to gravity, while the gyroscope 214 measures the rate of rotation around an axis. In particular, the IMU 210 may determine a phase of the gait cycle of the prosthetic ankle 200 based on data from the angular velocity measurement of the gyroscope 214, and the data from the accelerometer 212. The IMU 210 may take the form of or include a miniaturized MicroElectroMechanical System (MEMS) or a NanoElectroMechanical System (NEMS), as examples. Other types of IMUs may also be utilized. The IMU 210 may include other sensors, in addition to the accelerometer 212 and gyroscope 214, which may help to better determine position and/or help to increase autonomy of the prosthetic ankle 200. Two examples of such sensors are magnetometers and pressure sensors. Other examples are also possible.

A limit switch 216 may be used as an interrupt to the microcontroller 202 when the end of travel has been reached for the stiffness regulator. Such a configuration acts as a safety shutoff to keep from over torqueing the motor 206. Further, the limit switch 216 may be used to calibrate the prosthetic ankle 200, by determining where the top and bottom positions are in order to calculate the position of the stiffness regulator based on threaded rod rotations.

The microcontroller 202 may output data to two devices. First, the microcontroller 202 may output data to a SD card writer to track all sensor data from the prosthetic ankle for future evaluation. Such information may be used to determine future actions of the prosthetic ankle 200. The microcontroller 202 may be further configured to output signals to the motor speed controller 220 to power the motor 206 based on the received one or more inputs. The microcontroller 202 may determine where the stiffness regulator should be positioned and send the appropriate speed signals to the motor speed controller 220 using feedback from the motor encoder 208 to ensure that the proper location is reached.

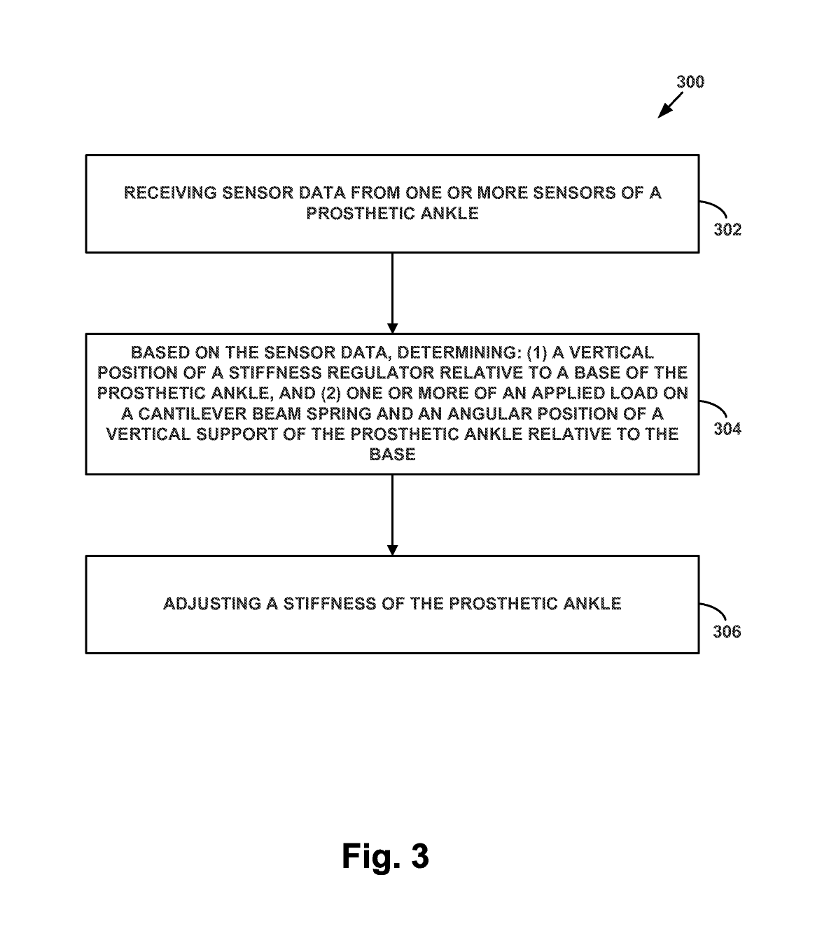

FIG. 3 is a block diagram of an example method for adjusting a stiffness of a prosthetic ankle in response to applied moments. Method 300 shown in FIG. 3 presents an embodiment of a method that could be used by the prosthetic ankle 100 of FIG. 1A, the prosthetic ankle 150 of FIG. 1B, or the prosthetic ankle 200 of FIG. 2, as examples. Method 300 may include one or more operations, functions, or actions as illustrated by one or more of blocks 302-306. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In addition, for the method 300 and other processes and methods disclosed herein, the block diagram shows functionality and operation of one possible implementation of present embodiments. In this regard, each block may represent a module, a segment, or a portion of program code, which includes one or more instructions executable by a processor or computing device for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (RO), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

In addition, for the method 300 and other processes and methods disclosed herein, each block in FIG. 3 may represent circuitry hat is wired to perform the specific logical functions in the process.

Initially, at block 302, the method hod 300 includes receiving data from one or more sensors of a prosthetic ankle. The prosthetic ankle may include any of the features described above in relation to FIGS. 1A-2. The one or more sensors may include one or more of a gyroscope, an accelerometer, an angular position sensor, an encoder, and a limit switch coupled to at least one component of a prosthetic ankle, including a base of the prosthetic ankle, a vertical support of the prosthetic ankle or a prosthetic foot coupled to the prosthetic ankle.

At block 304, the method 300 includes determining a vertical position of the stiffness regulator relative to the base of the prosthetic ankle, and one or more of an applied load on the cantilever beam spring and an angular position of the vertical support of the prosthetic ankle relative to the base. Such a determination is based on the received sensor data. In particular, the angular position of the vertical support relative to the base may be determined by sensor data from an angular position sensor coupled to the base of the prosthetic ankle, for example. The vertical position of the stiffness regulator relative to the base may be determined based on the number of rotations of the motor using a motor encoder, by relating the rotations of the motor to rotations of the threaded rod based on known gear ratios between the two components. Using the determined angular position of the prosthetic ankle and the position of the stiffness regulator, the applied load on the cantilever beam spring may be determined. The cantilever beam spring may also include one or more load cells to determine applied load. Example load cells may include a multi-axis load cell that includes strain gauges on multiple surfaces to sense forces along multiple axes. Other examples are possible as well.

At block 306, the method 300 includes adjusting a stiffness of the prosthetic ankle. In one example, the stiffness of the prosthetic ankle may be adjusted by moving the stiffness regulator along a threaded rod disposed in the vertical support in a direction away from the base to decrease stiffness, or moving the stiffness regulator along the threaded rod in a direction toward the base to increase stiffness. Other examples for adjusting the stiffness of the prosthetic ankle are possible as well.

In a particular example, the method may further include determining that the applied load on the cantilever beam spring exceeds a threshold, and responsively adjusting the stiffness of the prosthetic ankle according to a predetermined reaction protocol. For example, during straight line walking on relatively even terrain, the prosthetic ankle may operate with a moderate stiffness. However, while in operation a sudden high load on the cantilever beam spring may be encountered by treading on uneven terrain, or by turning a corner, or other non-straight line movements. In such a scenario, it may be desirable to cause the prosthetic ankle to stiffen to prevent over rotation of the vertical support relative to the base. As such, the prosthetic ankle may detect the high applied load, and responsively move the stiffness regulator in a direction away from the base at a first rate to achieve a predetermined decrease in stiffness based on a predetermined reaction protocol. After achieving the predetermined decrease in stiffness, the prosthetic ankle may move the stiffness regulator in a direction towards the base at a second rate to achieve a predetermined increase in stiffness based on the predetermined reaction protocol. The first rate may be faster than the second rate, since the prosthetic ankle may need to quickly respond to the high load during the heel strike phase of the gait cycle, and can then stiffen slowly during a swing phase of the gait cycle. Other example reaction protocols are possible as well.

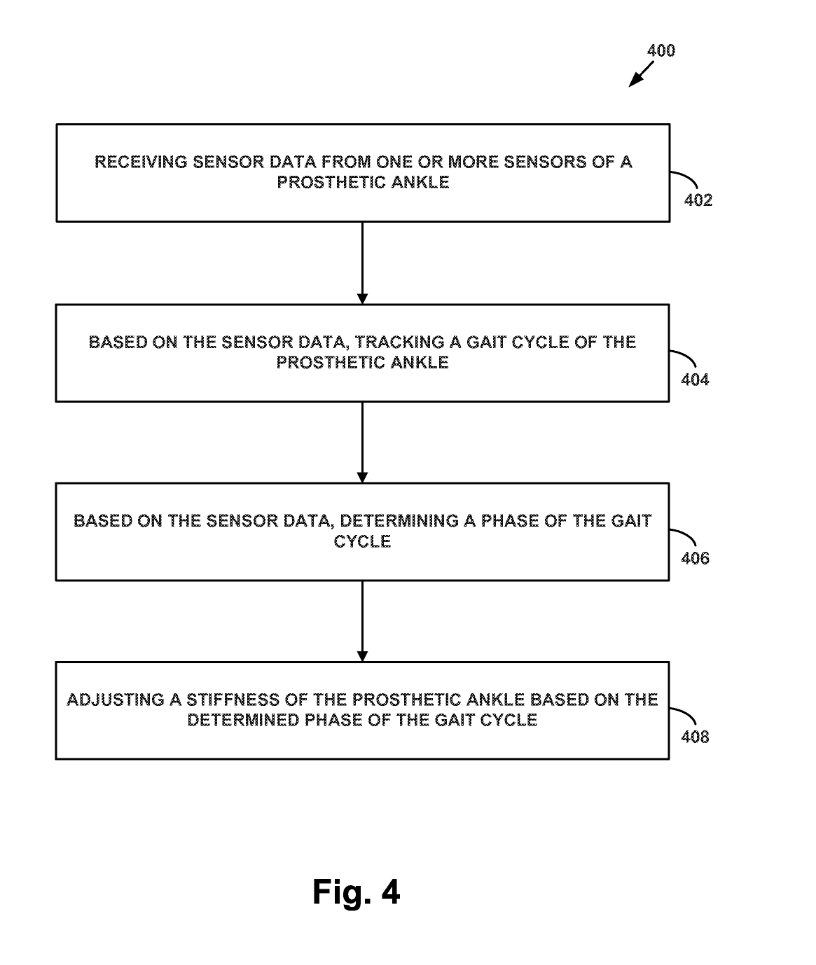

FIG. 4 is a block diagram of another example method for adjusting a stiffness of a prosthetic ankle based on a determined phase in a gait cycle. Method 400, shown in FIG. 4, presents an embodiment of a method that could be used by the prosthetic ankle 100 of FIG. 1A, the prosthetic ankle 150 of FIG. 1B or the prosthetic ankle 200 of FIG. 2, as examples. Method 400 may include one or more operations, functions, or actions illustrated by one or more of blocks 402-408. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

Initially, at block 402, the method 400 includes receiving sensor data from one or more sensors of a prosthetic ankle. The prosthetic ankle may include any of the features described above in relation to FIGS. 1A-2. As described above, the one or more sensors may include one or more of a gyroscope, an accelerometer, an angular position sensor, an encoder, and a limit switch coupled to at least one component of a prosthetic ankle, including a base of the prosthetic ankle, a vertical support of the prosthetic ankle or a prosthetic foot coupled to the prosthetic ankle.

At block 404, the method 400 includes tracking a gait cycle of the prosthetic ankle based on the sensor data. The gait cycle of the prosthetic ankle may be tracked using an IMU including an accelerometer and a gyroscope. The gyroscope may determine an angular velocity of the prosthetic ankle, while the accelerometer may detect the orientation of the prosthetic ankle with respect to gravity. At block 406, the method 400 includes determining a phase of the gait cycle based on the sensor data. In particular, based on the received data from the IMU, the prosthetic ankle may determine the phase of the gait cycle. The phase of the gait cycle may include one of a heel strike phase, a mid-stance phase, a toe-off phase, and a swing phase.

At block 408, the method 400 includes adjusting a stiffness of the prosthetic ankle based on the determined phase of the gait cycle. As described above, the stiffness of the prosthetic ankle may be adjusted by moving the stiffness regulator along a threaded rod disposed in the vertical support in a direction away from the base to decrease stiffness, or moving the stiffness regulator along the threaded rod in a direction toward the base to increase stiffness. Other examples for adjusting the stiffness of the prosthetic ankle are possible as well.

The prosthetic ankle may include a predetermined stiffness profile for each phase of the gait cycle. For example, adjusting the stiffness of the prosthetic ankle based on the determined phase of the gait cycle may include increasing the stiffness of the prosthetic ankle from the heel strike phase to the mid-stance phase, and decreasing the stiffness of the prosthetic ankle from the toe-off phase to the swing phase. Other example predetermined stiffness profiles are possible as well.

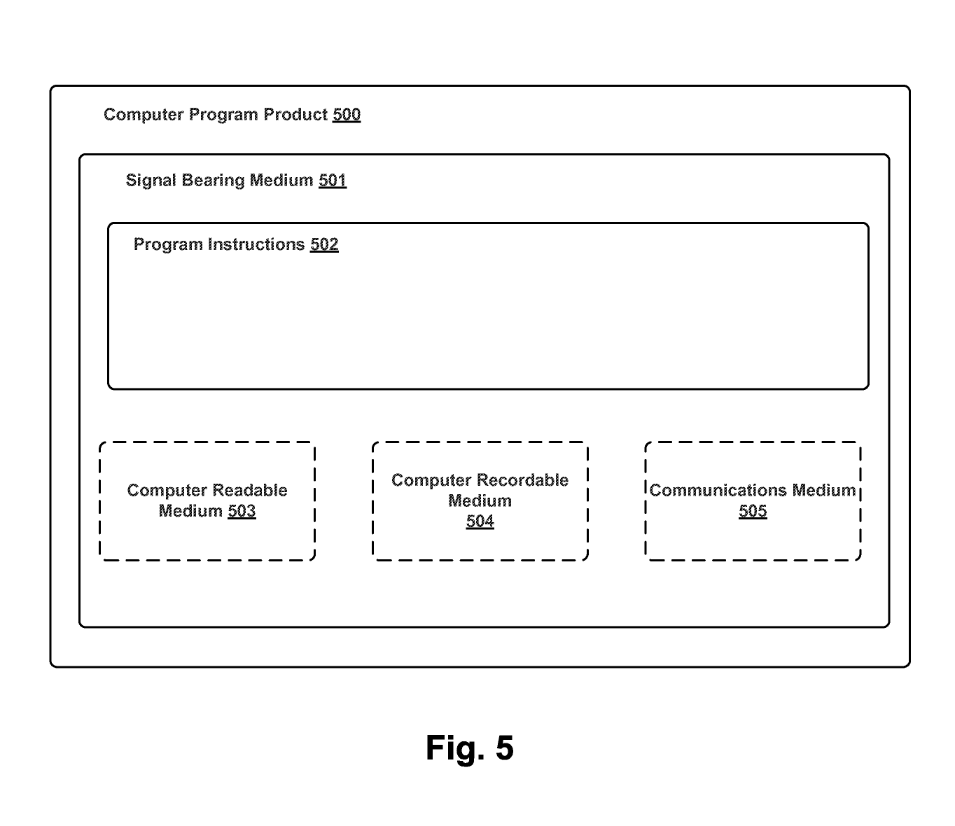

In some embodiments, the disclosed methods may be implemented as computer program instructions encoded on a non-transitory computer-readable storage media in a machine-readable format; or on other non-transitory media or articles of manufacture. FIG. 5 is a schematic illustrating a conceptual partial view of an example computer program product that includes a computer program for executing a computer process on a computing device, arranged according to at least some embodiments presented herein.

In one embodiment, the example computer program product 500 is provided using a signal bearing medium 501. The signal bearing medium 501 may include one or more programming instructions 502 that, when executed by one or more processors may provide functionality or portions of the functionality described above with respect to FIGS. 1-4. In some examples, the signal bearing medium 501 may encompass a computer-readable medium 503, such as, but not limited to, a hard disk drive, a Compact Disc (CD), a Digital Video Disk (DVD), a digital tape, memory, etc. In some implementations, the signal bearing medium 501 may encompass a computer recordable medium 504, such as, but not limited to, memory, read/write (R/W) CDs, R/W DVDs, etc. In some implementations, the signal bearing medium 501 may encompass a communications medium 505, such as, but not limited to, a digital and/or an analog communication medium (e.g., a fiber optic cable, a waveguide, a wired communications link, a wireless communication link, etc.). Thus, for example, the signal bearing medium 501 may be conveyed by a wireless form of the communications medium 505 (e.g., a wireless communications medium conforming with the IEEE 802.11 standard or other transmission protocol).

The one or more programming instructions 502 may be, for example, computer executable and/or logic implemented instructions. In some examples, a computing device such as the microcontroller 202 of FIG. 2 may be configured to provide various operations, functions, or actions in response to the programming instructions 502 conveyed to the computer program product 500 by one or more of the computer readable medium 503, the computer recordable medium 504, and/or the communications medium 505.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Since many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. A prosthetic ankle comprising:
a base;
a vertical support having a first end pivotally coupled to the base;
a first cantilever beam spring having a fixed end coupled to the base such that a length of the first cantilever beam spring in an unloaded position is substantially parallel to a longitudinal axis of the vertical support when the vertical support is in a neutral position; and
a stiffness regulator movably coupled to the vertical support, wherein the stiffness regulator has a platform that defines a first contact surface adjacent the first cantilever beam spring, and wherein the stiffness regulator pivots with the vertical support relative to the base.

2. (canceled)

3. The prosthetic ankle of claim 1, wherein the platform further defines a second contact surface adjacent the first cantilever beam spring, wherein the first contact surface is adjacent an outer surface of the first cantilever beam spring, and wherein the second contact surface is adjacent an inner surface of the first cantilever beam spring.

4. The prosthetic ankle of claim 3, wherein the first contact surface comprises at least one bearing adjacent the outer surface of the first cantilever beam spring, and wherein the second contact surface comprises at least one bearing adjacent the inner surface of the first cantilever beam spring.

5. The prosthetic ankle of claim 1, wherein the vertical support pivots relative to the base in a coronal plane of the prosthetic ankle.

6. The prosthetic ankle of claim 1, wherein the vertical support pivots between about zero degrees and about twenty-five degrees from the longitudinal axis of the vertical support in both an inverted direction and an everted direction.

7. The prosthetic ankle of claim 1, further comprising a threaded rod coupled to the first end of the vertical support and coupled to a second end of the vertical support.

8. The prosthetic ankle of claim 7, wherein the stiffness regulator comprises a threaded nut that is centrally disposed in the platform, wherein the threaded nut is coupled to the threaded rod such that the threaded rod extends through the platform of the stiffness regulator.

9. The prosthetic ankle of claim 7, further comprising a motor coupled to the threaded rod and configured to rotate the threaded rod relative to the stiffness regulator to move the stiffness regulator along the threaded rod between the first and second ends of the vertical support.

10. The prosthetic ankle of claim 1, further comprising:
one or more of a gyroscope, an accelerometer, an angular position sensor, an encoder, and a limit switch coupled to at least one of the base, the vertical support or a prosthetic foot.

11. The prosthetic ankle of claim 10, further comprising:
a microcontroller configured to receive one or more inputs from one or more of the gyroscope, the accelerometer, the angular position sensor, the encoder and the limit switch, wherein the microcontroller is configured to control the motor based upon the one or more inputs.

12. The prosthetic ankle of claim 1, further comprising:
a second cantilever beam spring having a fixed end coupled to the base such that a length of the second cantilever beam spring in an unloaded position is substantially parallel to a longitudinal axis of the vertical support when the vertical support is in the neutral position and such that the vertical support is disposed between the first and second cantilever beam springs.

13. The prosthetic ankle of claim 12, wherein the platform of the stiffness regulator defines a third contact surface adjacent the second cantilever beam spring.

14. The prosthetic ankle of claim 13, wherein the first contact surface comprises at least one bearing adjacent an outer surface of the first cantilever beam spring, and wherein the third contact surface comprises at least one bearing adjacent an outer surface of the second cantilever beam spring.

15. The prosthetic ankle of claim 13, wherein the first contact surface comprises at least one bearing adjacent an inner surface of the first cantilever beam spring, and wherein the third contact surface comprises at least one bearing adjacent an inner surface of the second cantilever beam spring.

16. The prosthetic ankle of claim 7, further comprising:
a guide rod coupled to the first end and the second end of the vertical support and disposed through a through-hole defined in the platform of the stiffness regulator.

17. The prosthetic ankle of claim 16, further comprising:
a linear bearing coupled to the guide rod and to the platform of the stiffness regulator.

18. The prosthetic ankle of claim 1, wherein the first cantilever beam spring includes a variable profile, such that a width of the first cantilever beam spring varies along the length of the first cantilever beam spring.

19. The prosthetic ankle of claim 1, wherein the vertical support has a first end pivotally coupled to the base via one of a pin joint, a hinge joint, a four bar linkage, or a ball and socket joint.

20. A method comprising:
receiving, by a computing device, sensor data from one or more sensors of a prosthetic ankle, wherein the prosthetic ankle comprises a vertical support having a first end pivotally coupled to a base, wherein the prosthetic ankle includes a cantilever beam spring having a fixed end coupled to the base such that a length of the first cantilever beam spring in an unloaded position is substantially parallel to a longitudinal axis of the vertical support when the vertical support is in a neutral position, and wherein the prosthetic ankle further includes a stiffness regulator movably coupled to the vertical support; and
(a) (i) based on the sensor data, determining, via the computing device, a vertical position of the stiffness regulator relative to the base and one or more of an applied load on the cantilever beam spring and an angular position of the vertical support relative to the base; and (ii) adjusting a stiffness of the prosthetic ankle; or
(b) (i) based on the sensor data, tracking, via the computing device, a gait cycle of the prosthetic ankle; (ii) based on the sensor data, determining, via the computing device, a phase of the gait cycle; and (iii) adjusting a stiffness of the prosthetic ankle based on the determined phase of the gait cycle.

21.-27. (canceled)

28. A non-transitory computer readable medium having stored therein instructions executable by a computer system to cause the computer system to perform functions, the functions comprising:
receiving sensor data from one or more sensors of a prosthetic ankle, wherein the prosthetic ankle comprises a vertical support having a first end pivotally coupled to a base, wherein the prosthetic ankle includes a cantilever beam spring having a fixed end coupled to the base such that a length of the first cantilever beam spring in an unloaded position is substantially parallel to a longitudinal axis of the vertical support in a neutral position, and wherein the prosthetic ankle further includes a stiffness regulator movably coupled to the vertical support; and
(a) (i) based on the sensor data, determining a vertical position of the stiffness regulator relative to the base and one or more of an applied load on the cantilever beam spring and an angular position of the vertical support relative to the base; and (ii) adjusting a stiffness of the prosthetic ankle; or
(b) (i) based on the sensor data, tracking a gait cycle of the prosthetic ankle; (ii) based on the sensor data, determining a phase of the gait cycle; and (iii) adjusting a stiffness of the prosthetic ankle based on the determined phase of the gait cycle.

29.-33. (canceled)
